(19) United States
(12) Plant Patent Application Publication
(10) Pub. No.: US 2005/0235388 P1
(43) Pub. Date: Oct. 20, 2005

(54) 'Sweet Thing' magnolia

(50) MAGNOLIA VIRGINIANA; MAGNOLIA

(76) Inventors: Fernando Campbell Boyd III, Morrison, TN (US); George L. Dodson III, Murfreesboro, TN (US)

Correspondence Address: Jason L. Hornkohl, 3712 Woodmont Lane, Nashville, TN, 37215, US

(21) Appl. No.: 10/824,299

(22) Filed: Apr. 15, 2004

(51) Int. Cl. A01H 5/00; C12Q 1/68; A61K 7/06

(52) U.S. Cl. PLT/223

(57) ABSTRACT

A new and distinct Magnolia virginiana var. australis cultivar, named ‘Sweet Thing’ Magnolia that is characterized by its distinct dwarf, bush-like growth habit, slender, olive green foliage (which remain--even in winter), fragrant flowers and vigorous ability to withstand extreme variations in temperature and moisture.

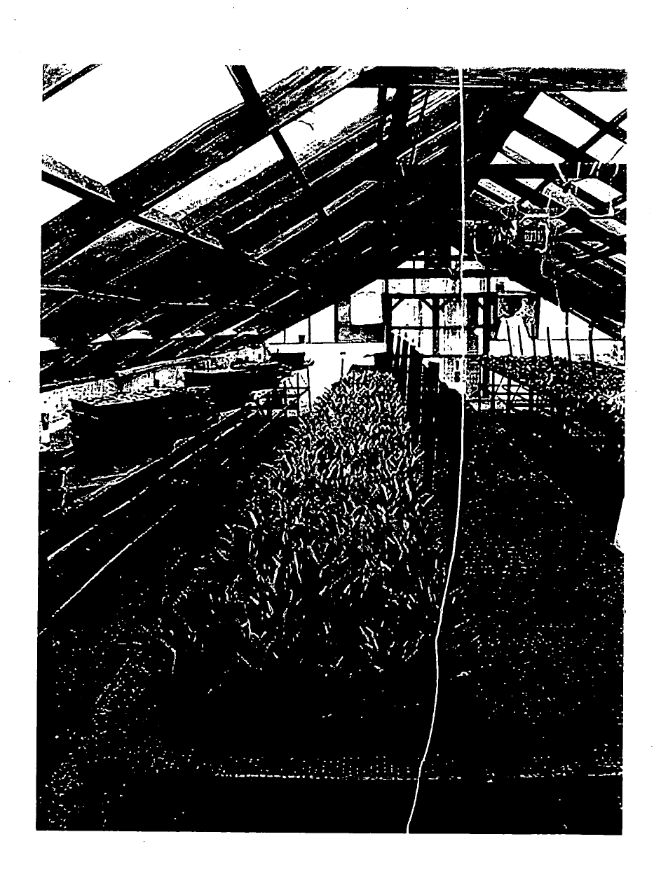

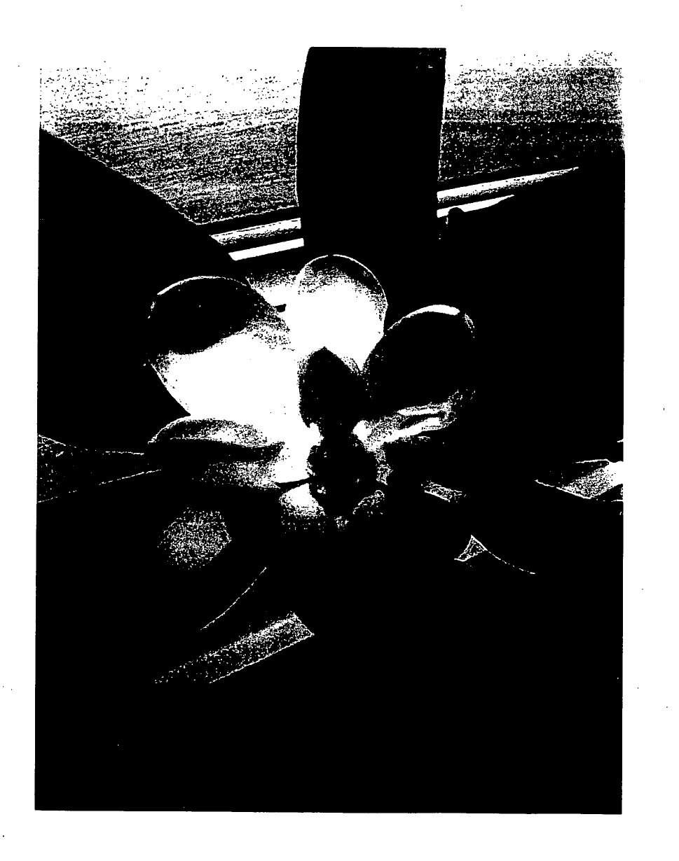

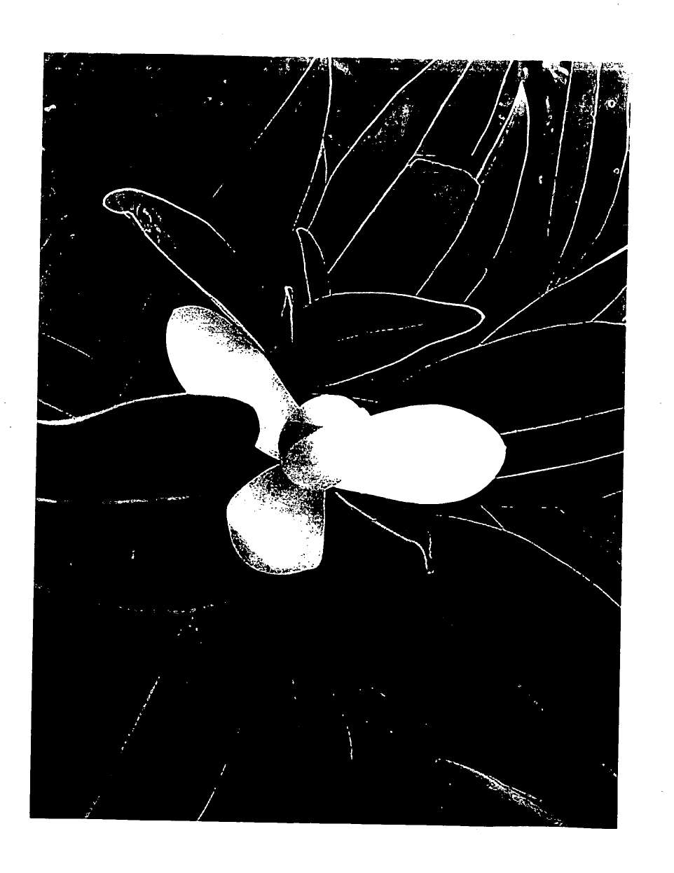

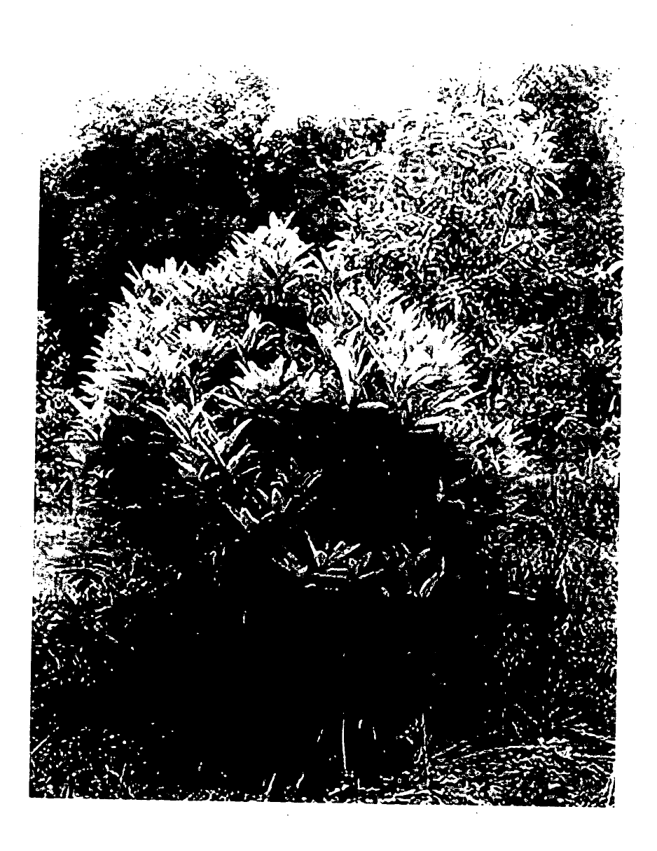

### FIELD OF THE INVENTION

The present invention comprises a new and distinct cultivar of Magnolia virginiana var. australis, and referred to by the cultivar name ‘Sweet Thing’.

### BACKGROUND OF THE INVENTION

This new dwarf cultivar of Magnolia virginiana var. australis, the ‘Sweet Thing’ magnolia, was originally discovered by George L. Dodson III, in a group of Magnolia virginiana var. australis seedlings planted at Sleepy Hollow Nursery, 3506 Harrison Ferry Road, McMinnville, Tenn. 37110, in 1990. Those responsible for this new cultivar are George L. Dodson III of Sleepy Hollow Nursery and Fernando Campbell Boyd III of Boyd Nursery. The initially discovered tree is still growing in a cultivated area at the Sleepy Hollow Nursery.

It was immediately recognized that the new cultivar, ‘Sweet Thing’, was distinctively different in growth from the other seedlings in the block. It had an attractive, denser, more compact branch structure and a slower growth pattern. The other seedlings growing in this population were indicative of normal Magnolia virginiana var. australis with the exception of the selected individual. While the ‘Sweet Thing’ seedling is shrubby and dense in its growth habit, the other seedlings in the block are tall and leggy in growth. After 14 years, the other seedlings in the block are 20′ or more in height and approximately 8′ wide and very open in stature. In contrast, the ‘Sweet Thing’ cultivar is approximately 8′ tall by approximately 6′ wide. After monitoring the initial group for several years, it is apparent that while the typical Magnolia virginiana var. australis seedlings grow tall and leggy, the Magnolia virginiana var. australis ‘Sweet Thing’ remains small, compact, dense, and evergreen year after year.

As shown in the photographic drawings, the ‘Sweet Thing’ cultivar is very dense and full of foliage. The ‘Sweet Thing’ cultivar retains its foliage year round in the winter months in Zone 6b, which includes Middle Tennessee, as do typical Magnolia virginiana var. australis. As shown in more detail in the second and third photographic drawings, the foliage is an olive green on the upper surface and has a silvery sheen on the glaucous underside surface. The leaves are more lanceolate than is typical of the variety, measure approximately 7.6 cm to 16.5 cm in length and 2.85 cm to 4.52 cm in width, and are lustrous. They are not quite as long as, and slightly lighter green in color than, the typical Magnolia virginiana var. australis.

As shown in the fourth photographic drawing, the flower is cup-shaped, 10 to 12 cm across. The flower is white in color. The species typically has more of a creamy white flower. The flower is fragrant with a citrus scent and has 9 to 12 petals that and are approximately 4.5 cm to 5.5 cm long and 1.9 cm to 3.5 cm wide. The petals are obovate, separate, involute, entire margin, obtuse apex, and fused at the base. The blooming season is from August to October in Middle Tennessee and the blooms last about a week.

The ‘Sweet Thing’ cultivar is very winter hardy. The tree has proven to be evergreen in a Middle Tennessee climate Zone 6b (USDA Plant Hardiness Zone Map). However, in the severe winter of 1996, the parent plant kept most of its leaves when temperatures reached −10° F. (with a wind chill of −17° F.). In contrast, the other Magnolia virginiana var. australis in the initial group of seedlings lost most of their leaves, and the low temperatures damaged some of the trees. Thus, the ‘Sweet Thing’ cultivar appears to have a greater cold tolerance than the typical Magnolia virginiana.

The ‘Sweet Thing’ cultivar is also able to endure drastic changes in the moisture level. The ‘Sweet Thing’ cultivar has been successfully grown without any irrigation. In addition, the parent plant is planted close to a river, and has been completely submerged underwater, because of periodic flooding, at least four times since 1990. Thus, the ‘Sweet Thing’ cultivar thrives in moist soil conditions while tolerating the hot dry conditions of summer.

The ‘Sweet Thing’ cultivar has been successfully propagated by asexual propagation. The proven means of asexual propagation has been rooted cuttings. The propagation from the original ‘Sweet Thing’ Magnolia tree began in 1999 at Boyd Nursery. It has been successfully propagated through at least four generations of asexual reproduction, with the highest rooting percentage coming from cuttings taken from the newest generation. The ‘Sweet Thing’ Magnolia has retained its outstanding unique features throughout each generation of new plants. Each generation has been stable, and reproduced true-to-type plants each and every time the plant has been propagated.

The unique appearance and growth pattern of the ‘Sweet Thing’ cultivar make it well suited for a variety of landscaping uses. It can be used as an evergreen shrub or planted close together to create a novel and attractive hedge to obscure certain areas from view. It is also well suited for use as a foundation plant for larger buildings or in areas that are not large enough for a typical Magnolia virginiana. Since the ‘Sweet Thing’ Magnolia is evergreen with attractive, lustrous, olive green foliage, flowers with a nice fragrance, and is dwarf, it should be a welcome new landscape plant for small and large gardens or various landscape situations. In addition, the unique and attractive ‘Sweet Thing’ Magnolia will make a great show piece for those desiring a rare or unusual Magnolia virginiana var. australis specimen.

### SUMMARY OF THE INVENTION

The following characteristics in combination distinguish the new tree named ‘Sweet Thing’ from other cultivars of Magnolia virginiana var. australis.

1. The ‘Sweet Thing’ cultivar is a dwarf variant of the Magnolia virginiana var. australis that has a smaller more bush-like appearance. Young asexually propagated trees, like the initially discovered tree, all tend to grow with a multi-stem trunk. Thus, it is well suited for landscaping applications and areas where a typical Magnolia virginiana is too large.

2. The ‘Sweet Thing’ Magnolia has a longer, more slender leaf than a typical Magnolia virginiana that adds to its bush-like appearance. In addition, the slender leaves pose less of a clean up problem than the large, hard leaves of a typical Magnolia.

3. The leaf is a lighter green than a typical Magnolia virginiana which contributes to the ‘Sweet Thing’ Magnolia's distinctive and pleasing appearance.

4. The ‘Sweet Thing’ cultivar is more tolerant of extreme cold than a typical Magnolia virginiana var. australis, retaining its foliage year round in a Middle Tennessee climate despite temperatures of −10° F. (with a wind chill of −17° F.).

5. The ‘Sweet Thing’ cultivar is able to endure drastic changes in the moisture level. The parent plant has been grown, and is thriving, without any irrigation and tolerates the hot dry conditions of summer. In addition, the parent plant has survived being repeatedly submerged by a nearby flooding river.

The ‘Sweet Thing’ cultivar has not been observed under all possible conditions and it is not known how the cultivar might respond to various climates.

### BRIEF DESCRIPTION OF THE DRAWINGS

The first photographic drawing shows the parent ‘Sweet Thing’ plant at fourteen years of age at its home at the Sleepy Hollow Nursery.

The second photographic drawing shows a close up of the foliage and an emerging bloom of the ‘Sweet Thing’ cultivar.

The third photographic drawing shows a close up of the foliage and a fully developed bloom of the ‘Sweet Thing’ cultivar.

The fourth photographic drawing shows a number of second generation ‘Sweet Thing’ rooted cuttings vigorously growing at Boyd's Nursery.

### DETAILED BOTANICAL DESCRIPTION

The following observations, measurements, and values describe the parent plant grown at Sleepy Hollow Nursery in McMinnville, Tenn. The actual appearance and characteristics of any individual will of course vary due to horticultural practices and local conditions. The tree used for the description is about 14 years old. Color references are made to The Royal Horticultural Society Colour Chart except where terms of ordinary significance are used.
Origin: Seedling from planted group of seedlings.
Parentage: Unnamed Magnolia virginiana var. australis.
Propagation. Asexual through cuttings.
Plant:
   Growth rate.—slow to medium, average 15 cm per year.
   Form.—Small multi-stem tree or bush.
   Shape.—oval to round.
   Height.—8 ft in 14 years.
   Spread.—6 ft in 14 years.
   Density.—Thick with foliage.
   Trunk size.—7″ diameter at the base of the trunk at ground level at 14 years.
   Bark.—(trunk): smooth, color is (197A to 199A RHS) textured with tiny (1 mm) raised oval lenticels.
   Stem.—On the new growth of the stem, the color is (144A-C RHS). There are five main stems on the parent plant. They range in caliper from 12″ to 2½″.
   Lenticels.—Tiny, but conspicuous, silver, slightly raised, oval, 1 mm.
   Branching arrangement.—Bush-like, multi-stemmed, ‘Sweet Thing’ can be trained to a single stem.
   Leaves.—Evergreen.
   Length.—Petiole 1.27-2.5 cm, average 2.1 cm; Lamina 7.6 cm to 16.5 cm in length and 2.85 cm to 4.52 cm in width.
   Width.—2.85 cm to 4.52 cm.
   Form.—lanceolate.
   Margin.—entire.
   Texture.—glossy above, silvery-white beneath.
   Quantity.—abundant.
   Color.—Upper side: light green (137A to 137B RHS). Lower side: glaucous (silvery-green) (188B to 188D RHS).
   Ribs and veins.—Pinnately veined with 12-16 nearly opposite pairs (151A RHS).
   Buds.—(Vegetative) terminal, silvery-white pubescence, narrow conical, curved, 3.18 mm to 5.00 mm in diameter by 19.05-28.57 mm long. About (192A to 192D RHS) in color. Lateral buds, silvery-white pubescence, narrow conical, curved, 2.54 mm to 3.75 mm in diameter by 3.15 mm to 12.7 mm long. About (192A to 192D RHS) in color.
Flowers:
   Dormant flower buds.—Terminal, silvery-white pubescence and about (192A to 192D RHS) in color, bluntly pointed and average 9.5 mm in diameter. They average 22 mm in length.
   Flower.—cup-shaped, 10 to 12 cm across, white on the upper surface (155B RHS), and white on the lower surface (155C RHS).
   Petals.—number between 9-12; 4.5-5.5 cm long, 1.9-3.5 cm wide; obovate, separate, involute, entire margin, obtuse apex, fused at base.
   Fragrance.—fragrant with a citrus scent.
   Blooming season.—from August to October in Middle Tennessee.
   Bloom duration.—about one week.
   Stamens.—3 to 4 mm long; abundant; white (155B RHS) before anthesis, brown after; self-fertile.
   Pollen amount.—Moderate.
   Pollen color.—Yellow.
   Fruit.—Subglobose to ellipsoid, light green to brown conelike aggregate of follicles with seeds dispersed throughout the follicles of the cone. Seeds are red in color (42A RHS).
Disease and pest resistance: No known susceptibility to diseases and pests common to Magnolia virginiana var. australis.

1. A new and distinct cultivar of Sweet Bay Magnolia tree named ‘Sweet Thing’ as illustrated and described herein.
